(19) United States
(12) Patent Application Publication
(10) Pub. No.: US 2013/0317390 A1
(43) Pub. Date: Nov. 28, 2013

(54) CYTOLOGICAL SAMPLE ACQUISITION DEVICE AND METHOD

(71) Applicants: Michael J. Levy, Rochester, MN (US); Christopher J. Gostout, Rochester, MN (US)

(72) Inventors: Michael J. Levy, Rochester, MN (US); Christopher J. Gostout, Rochester, MN (US)

(73) Assignee: MAYO FOUNDATION FOR MEDICAL EDUCATION AND RESEARCH, Rochester, MN (US)

(21) Appl. No.: 13/982,137

(22) PCT Filed: Jan. 25, 2012
(86) PCT No.: PCT/US2012/022504

§ 371 Date: Aug. 15, 2013

Related U.S. Application Data
(60) Provisional application No. 61/436,837, filed on Jan. 27, 2011.

(51) Int. Cl. A61B 10/04

(52) U.S. Cl. CPC A61B 10/04
USPC 600/563; 600/562; 600/570

(57) ABSTRACT

A tissue collection device and method. One embodiment of the device includes a body formed by an elongated member, a pair of inflatable balloons at spaced-apart locations on the body, and one or more collection structures on the body. Inflation lumens extending through the body and coupled to the balloons cause the balloons to move between collapsed and expanded states. The collection structures collect a tissue sample from a collection region between the balloons. In another embodiment the collection structures include one or more of an irrigation lumen and an aspiration lumen having one or more ports opening into the collection region.

### TECHNICAL FIELD

The invention relates generally to a surgical instrument and a surgical method performed with the instrument. In particular, the invention is a device for acquiring tissue samples for cytology analyses and surgical procedures for using the tools to collect cytological samples.

### BACKGROUND OF THE INVENTION

Devices and methods for collecting tissue samples from a patient for cytological analysis in a laboratory are generally known. During a cytological sample acquisition procedure a sample collection device is typically delivered to a sample site within a body lumen through the use of an endoscope and/or catheter. Following the collection of the tissue sample the device is withdrawn. By way of example, the Sahatjian U.S. Pat. No. 5,409,012 and the Parasher U.S. Pat. No. 5,535,756 disclose instruments and surgical procedures for collecting cytological samples. There remains, however, a continuing need for enhanced cytological acquisition and sampling technologies.

### SUMMARY

One embodiment of the invention is a tissue collection device that includes a body, first and second diametrically expandable members at spaced-apart locations on the body, one or more actuating structures, and one or more collection structures on the body. The actuating structures cause the expandable members to move between expanded and collapsed states. The collection structures collect a tissue sample from a collection region between the first and second expandable members.

In another embodiment the expandable members include balloons, and the actuating structures include inflation lumens. The body is an elongated member and the balloons are fixedly mounted to the elongated member. The collection structures include one or more of an irrigation lumen and an aspiration lumen having one or more ports opening into the collection region.

Another embodiment of the invention is a method for acquiring a cytological sample using the tissue collection device. The method includes delivering the collection device to a sample site through a body lumen, expanding the first and second expandable members, and collecting a tissue sample from the collection site between the first and second expandable members. The expandable members can be collapsed, and the collection device withdrawn.

### BRIEF DESCRIPTION OF THE DRAWINGS

FIG. 1A is an illustration of a sample collection device in accordance with one embodiment of the invention in a delivery state.

FIG. 1B is an illustration of the sample collection device shown in FIG. 1A in a collection state.

FIG. 2 is an illustration of a cytological sample acquisition method in accordance with one embodiment of the invention for using the sample collection device shown in FIG. 1A.

FIG. 3 is an illustration of a sample collection device in accordance with a second embodiment of the invention in a deployed state at a sample site within a body lumen.

FIG. 4 is an illustration of a sample collection tool in accordance with another embodiment of the invention.

FIG. 5 is an illustration of the sample collection tool shown in FIG. 4 in the operational state.

### DETAILED DESCRIPTION

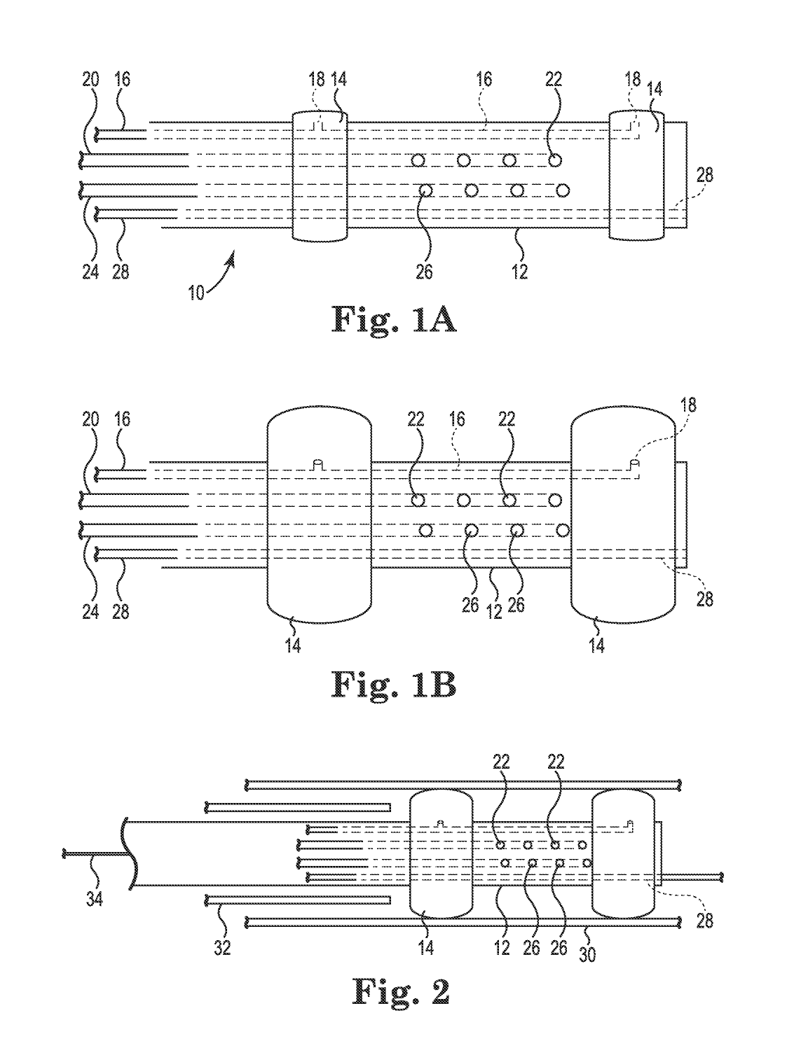

A cytological sample collection device 10 in accordance with one embodiment of the present invention is illustrated generally in FIGS. 1A and 1B. As shown, device 10 includes an elongated delivery catheter body 12 and a spaced-apart pair of diametrically expandable members such as balloons 14. One or more expansion structures such as inflation lumen 16 extend through the body 12 and connect to the expandable members. The illustrated embodiment has one inflation lumen 16 with a pair of inflation ports 18 that open into both balloons 14. Other embodiments (e.g., device 110 described below) have a separate expansion structure coupled to each expandable member. FIG. 1A shows collection device 10 in a delivery state with balloons 14 deflated or diametrically collapsed, while FIG. 1B shows the device in a collection state with the balloons inflated or diametrically expanded. An irrigation lumen 20 extends through the body 12 and communicates with one or more irrigation ports 22 located on the outside of the body 12 between the balloons 14. Similarly, an aspiration lumen 24 extends through the body 12 and communicates with suction ports 26 located on the outside of the body between the balloons 14. Other embodiments (not shown) have one lumen connected to one or more ports that are used for both irrigation and aspiration. The illustrated embodiment of collection device 10 also includes an optional guide wire lumen 28. Still other embodiments of the invention (not shown) have monorail structures for receiving and cooperating with an external guide wire. The size of the delivery catheter body 12 can vary depending on the application of the collection device 10 and the number and size of structures (e.g., lumens 20, 24 and 28) that are incorporated into the body. For example, the delivery catheter body 12 can be 1O F-11.5 F, or even larger as allowed by the introduction of larger channel endoscopes or duodenoscopes. Similarly, the size of the balloons 14 can vary based on factors such as the application of the collection device 10. Yet other embodiments of the invention (not shown) have one or more accessory lumens extending through the body to enable other devices and tools to access the collection zone space between the balloons 14.

A cytological tissue acquisition procedure in accordance with one embodiment of the invention can be described with reference to FIG. 2. With the device 10 in the delivery state (e.g., with balloons 14 collapsed), the collection device 10 is delivered through a body lumen 30 to a sample site at which it is desired to collect a tissue sample. In the illustrated embodiment the collection device 10 is delivered through an endoscope 32 and over a guide wire 34. In other embodiments of the invention the collection device 10 is delivered to the sample site using alternative approaches, such as either an endoscope or a guide wire, or without the use of either an endoscope or a guide wire. The collection device 10 is generally positioned with the first and second or distal and proximal balloons 14 on opposite ends of the sample site. The balloons 14 are then expanded to minimize the risk of loss of the collected specimen. In the illustrated embodiment the balloons 14 are inflated through the delivery of inflation fluid (e.g., saline or gas) from a source (not shown) through inflation lumen 16.

After the balloons 14 are expanded, saline or other collection fluid is delivered from a source (not shown) to the sample site through the irrigation lumen 20 and irrigation ports 22. The collection fluid can, for example, be delivered under high pressure. Alternatively or in addition, exfoliating agents or other substances that enhance cell release and collection can be delivered through the irrigation ports 22. The volume of the collection zone between the balloons 14 can be completely or partially filled with the collection fluid in this manner. After delivery to the sample site, the collection fluid (and tissue sample that has been suspended in the collection fluid) is aspirated through the suction ports 26 and aspiration lumen 24. In one embodiment of the invention 100-200 ml of collection fluid is retrieved, although more or less fluid can be used in other embodiments. In the illustrated embodiment having a separate irrigation lumen 20 and aspiration lumen 24, the collection fluid can be infused and aspirated separately or simultaneously. In other embodiments (not shown) having a common aspiration and collection lumen, the collection fluid is aspirated after being infused. In still other embodiments of the invention other tools or devices are delivered to the sample site through an accessory lumen in the collection device 10. For example, a brush or an abrasive or scraping tool can be inserted through the accessory lumen and used to enhance the removal of tissue from the sample site.

Following the sample collection and the removal of any tools inserted through the collection device 10, the balloons 14 can be collapsed and the collection device returned to the delivery state and withdrawn from the body lumen 12. The collection fluid will be processed and the samples analyzed in known or otherwise conventional manners (e.g., the fluid is centrifuged and the cell pellet analyzed).

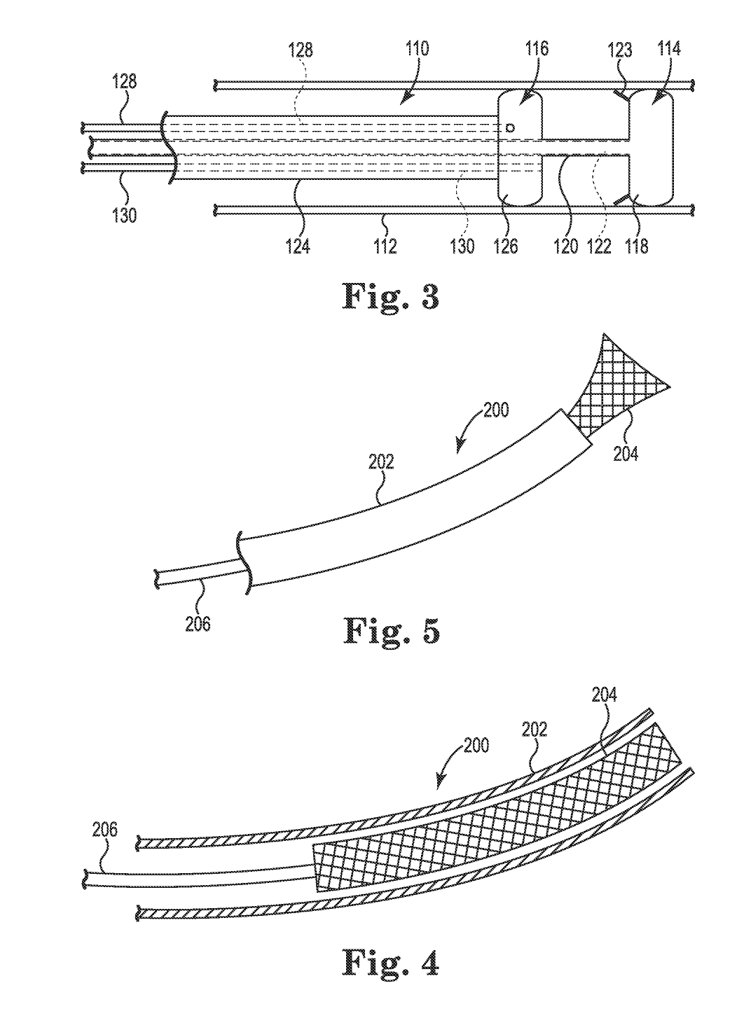

FIG. 3 is an illustration of a cytological sample collection device 110 in accordance with another embodiment of the invention following delivery to a sample site within a body lumen 112. Collection device 110 is a multi-part device including distal component 114 and proximal component 116. Distal component 114 includes a balloon 118 and a body member 120 having an inflation lumen 122 in fluid communication with the balloon. A scraper or abrasive edge 123 is located on the balloon in the illustrated embodiment. Still other embodiments (not shown) have a sharp rim or edge, brush bristles, coatings, sticky surface or other structures on the surface of one or both balloons 118 and 126 to aid in the capture of cells. Other embodiments do not have this cell capture enhancement structure. Proximal component 116 includes a catheter body 124 and a balloon 126 on the body. An inflation lumen 128 extends through the body 124 into fluid communication with the balloon 126. An irrigation/aspiration lumen 130 extends through the body 112 and balloon 126 so as to be in fluid communication with a space between balloons 118 and 126. As shown, the body member 120 of distal component 114 extends through the balloon 126 and body 124 of the proximal component 116.

During a cytological sample acquisition procedure the proximal component 116 is delivered to the sample site with the balloon 126 collapsed. The proximal component balloon 126 is then inflated by the introduction of fluid through the inflation lumen 128. The diametrically expanded balloon 126 thereby creates a seal that will prevent retrograde fluid flow through the body lumen 112. After placement of the proximal component 116, the distal component 114, in the delivery state, is inserted through the proximal component and the balloon 118 advanced to a position beyond balloon 116 of the proximal component. Balloon 118 of the distal component is then inflated by the introduction of fluid through inflation lumen 122.

After the collection device 110 is positioned at the sample site, it can be operated one of several different manners to collect a tissue sample. For example, collection fluid can be introduced and withdrawn through the irrigation/aspiration lumen 130 in the manner described above in connection with collection device 10. Alternatively, by manipulating the member 120, balloon 118 can be moved in a rotational or reciprocal manner to mechanically disrupt cells before the collection fluid is introduced and withdrawn to collect the sample. This mechanical disruption of the tissue at the sample site can be enhanced by the abrasive edge 123 on the balloon 118. In another alternative approach, the balloon 118 is deflated and the distal component 114 removed from the sample site before suction is applied to the irrigation/aspiration lumen 130 to remove the tissue sample. In another approach the balloon 118 in the distal component 114 can be filled with cell preservative solution (e.g., through the inflation lumen 122) and overfilled to burst and release the preservative. The preservative dispensing approach can but need not be done before the mechanical disruption but will typically be done before aspiration of the tissue sample. Following the collection of the tissue sample the components 114 and/or 116 can be returned to their delivery state and withdrawn from the sample site. The method described above can, for example, be used for indications in the bile duct, pancreatic duct, pancreatic cysts, gall bladder, urethra and uterine system.

Alternative versions of collection device 110 can include one or more other features described in connection with collection device 10. For example, collection device 110 can include separate irrigation and aspiration lumens and/or a guide wire lumen and/or one or more accessory lumens. Collection device 110 can also be delivered using any of the approaches described above in connection with collection device 10 (e.g., through an endoscope and/or over a guide wire). Although the embodiments of collection devices 10 and 110 described above have balloons, other diametrically expandable structures and associated expansion members can be used. For example, other embodiments (not shown) have a diametrically expandable umbrella-like structure and a mechanical expansion member.

FIG. 4 is an illustration of one embodiment of a tool 200 that can be used in connection with the collection devices 10 and 110 and the sample acquisition procedures described above. Tool 200 is shown in a retracted, delivery state in FIG. 4. FIG. 5 is an illustration of the tool 200 in an extended, operational state. As shown, tool 200 includes a tubular delivery sheath 202 and an expandable disruption member 204. An actuating member 206 is connected to the disruption member 204 and extends from the delivery sheath 202 to a position or structure (e.g. a handle or motor) at which it can be actuated. In the illustrated embodiment the disruption member 204 is a tubular braided mesh of resilient wires that function in a manner similar to a self-expanding stent or so-called Chinese finger trap, though plastic, polymers, or other materials could also be used. In some cases, the disruption member 204 can be balloon-inflatable. Other embodiments of tool 200 have other radially or diametrically expandable disruption members. In yet another embodiment of the invention disruption can be performed using a tool comprising a solid tubular member (e.g., similar to a plastic biliary stent) having studs, ribs, bristles or other disruption members on its outer surface.

In one embodiment of the invention, tool 200 is used in connection with the collection devices 10 and 110 described above. In particular, with the disruption member 204 retained in a retracted position within the sheath 202, the tool 200 is inserted through an accessory lumen in the collection device and advanced through the accessory lumen to position the distal end of the tool at the sample site. By manipulating the actuating member with respect to the delivery sheath 202 the disruption member 204 is extended from the tool 200. The delivery sheath 202 and/or the actuating member 206 can then be actuated (e.g., moved in a rotating or reciprocal manner) while the disruption member 204 is in contact with tissues at the sample site. By this motion the disruption member 204 enhances the mechanical release of cells from the sample site. Following the use of the tool 200 in this manner, the disruption member 204 can be retracted into the sheath 202 and the tool removed from the collection device and body lumen. In alternative embodiments (not shown) a motor is coupled to the disruption member 206 (e.g., through the actuating member 206) to rotate the disruption member during the sample acquisition process. In still other embodiments of the invention, tool 200 can be used with other tissue removal methods and/or with other devices. For example, the tool 200 can be delivered to a sample site through an endoscope or over a guide wire, and cells released by the abrasion process collected within the sheath 202 when the disruption member 204 is retracted into the sheath.

Although the present invention has been described with reference to preferred embodiments, those skilled in the art will recognize that changes can be made in form and detail without departing from the spirit and scope of the invention.

1. A tissue collection device, including:
a body;
first and second diametrically expandable members at spaced-apart locations on the body;
one or more actuating structures for causing the expandable members to move between expanded and collapsed states; and
one or more collection structures on the body for collecting a tissue sample from a collection region between the first and second expandable members.

2. The tissue collection device of claim 1 wherein:
the first and second expandable members include balloons; and
the one or more actuating structures include inflation lumens.

3. The tissue collection device of claim 1 wherein the one or more collection structures includes one or more of an irrigation lumen and an aspiration lumen having one or more ports opening into the collection region.

4. The tissue collection device of claim 1 wherein the body is an elongated member and the first and second expandable members are fixedly mounted to the elongated member.

5. The tissue collection device of claim 1 wherein the body includes:
a proximal body member having a lumen, wherein the first expandable member is mounted to the proximal body member;
a distal body member, wherein the second expandable member is mounted to the distal body member; and
wherein the distal body member and second expandable member can be inserted though the lumen in the proximal body member and the second expandable member located distally of the first expandable member.

6. A method for acquiring a cytological sample using a tissue collection device of the type including a body, first and second diametrically expandable members at spaced-apart locations on the body, one or more actuating structures for causing the expandable members to move between expanded and collapsed states, and one or more collection structures on the body for collecting a tissue sample from a collection region between the first and second expandable members, comprising:
delivering the proximal body member in a delivery state through a body lumen and positioning the first expandable member at a sample site;
delivering the distal body member through the lumen in the proximal body member, and positioning the second expandable member distal to the first expandable member at the sample site;
diametrically expanding the first and second expandable members;
collecting a tissue sample from the sample site between the first and second expandable members;
collapsing the first and second expandable members; and
withdrawing the proximal and distal body members.

7. The method of claim 6 including expanding the first expandable member before expanding the second expandable member.

8. The method of claim 6 wherein collecting a tissue sample includes irrigating the sample site with fluid and aspirating the fluid.

9. The method of claim 6 wherein collecting a tissue sample includes moving the second expandable member.

10. The method of claim 6 and further including inserting a tissue releasing tool into the space between the first and second actuating members and actuating the tool to abrade tissue at the sample site.

11. A method for acquiring a cytological sample using a tissue collection device of the type including a body, first and second diametrically expandable members at spaced-apart locations on the body, one or more actuating structures for causing the expandable members to move between expanded and collapsed states, and one or more collection structures on the body for collecting a tissue sample from a collection region between the first and second expandable members, comprising:
delivering the collection device to a sample site through a body lumen;
expanding the first and second expandable members;
collecting a tissue sample from the collection site between the first and second expandable members;
collapsing the first and second expandable members; and
withdrawing the collection device.

12. The method of claim 11 including independently expanding the first expandable member and the second expandable member

13. The method of claim 11 including simultaneously expanding the first and second expandable members.

14. The method of claim 11 wherein collecting a tissue sample includes irrigating the sample site with fluid and aspirating the fluid.

15. The method of claim 11 and further including inserting a tissue releasing tool into the space between the first and second actuating members and actuating the tool to abrade tissue at the sample site.
